(19) United States
(12) Patent Application Publication
(10) Pub. No.: US 2024/0270686 A1
(43) Pub. Date: Aug. 15, 2024

(54) METHOD FOR PREPARING MERCAPTANS WITH SULFHYDROLYSIS OF PURIFIED DIALKYL SULFIDES

(71) Applicant: ARKEMA FRANCE, Colombes (FR)

(72) Inventors: Georges FREMY, Lacq (FR); Hélori SALEMBIER, Lacq (FR); Guillaume LAUDUMIEY, Lacq (FR); Joaquin LACAMBRA, Lacq (FR); Eric LAMANT, Lacq (FR)

(73) Assignee: ARKEMA FRANCE, Colombes (FR)

(21) Appl. No.: 18/567,183

(22) PCT Filed: Jun. 20, 2022
(86) PCT No.: PCT/FR2022/051193

§ 371 Date: Dec. 5, 2023

(30) Foreign Application Priority Data
Jun. 21, 2021 (FR) FR2106568

(51) Int. Cl. C07C 319/08; C07C 319/28

(52) U.S. Cl. CPC C07C 319/08; C07C 319/28

(57) ABSTRACT

The present invention relates to a process for preparing at least one mercaptan, comprising the following steps:
A) H₂S and at least one alcohol are introduced into a first reactor;
B) the H₂S and said at least one alcohol are reacted to obtain an outlet stream comprising at least one mercaptan, at least one dialkyl sulfide and at least one dialkyl disulfide (DADS) and possibly unreacted H₂S;
C) said outlet stream obtained from step B) is separated into:
   a stream F1 comprising the mercaptan(s),
   a stream F2 comprising the dialkyl sulfide(s) and the DADS(s), and
   optionally a stream F3 comprising H₂S;
D) a purification step is performed on stream F2 in order to separate:
   a stream F2′ comprising the dialkyl sulfide(s); and
   the DADS(s);
E) stream F2′ is introduced with H₂S into a second reactor;
F) a sulfhydrolysis reaction of the dialkyl sulfide(s) with H₂S is performed to obtain an outlet stream F4 comprising said mercaptan(s), and possibly unreacted H₂S;
G) optionally, stream F4 obtained from step F) is recycled into step A).

The present invention relates to a process for preparing mercaptans, from at least one alcohol and hydrogen sulfide, involving a process for the sulfhydrolysis of purified dialkyl sulfides. Mercaptans are of great interest industrially and are currently in widespread use in the chemical industries, notably as starting materials in the synthesis of more complex organic molecules. For example, methyl mercaptan (CH₃SH) is used as a starting material in the synthesis of methionine, an essential amino acid for animal nutrition. Methyl mercaptan is also used in the synthesis of dialkyl disulfides, in particular in the synthesis of dimethyl disulfide (DMDS), a sulfiding additive for hydrotreating catalysts for petroleum fractions, among other applications.

Mercaptans, and in particular methyl mercaptan, are generally synthesized industrially by a known process starting from an alcohol and hydrogen sulfide at elevated temperature in the presence of a catalyst according to equation (1) below:

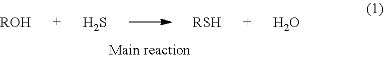

However, this reaction gives rise to the formation of byproducts, such as dialkyl sulfides (which are symmetrical in the case below) according to equation (2) below:

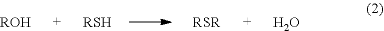

Furthermore, when the main reaction is performed in the presence of several alcohols, dissymmetrical dialkyl sulfides may also be obtained according to equations (3) and (4) below (example given with two alcohols):

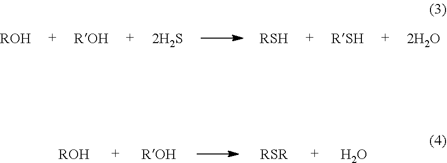

The symmetrical or dissymmetrical dialkyl sulfide byproducts are obtained in large amount industrially and are primarily sent for destruction. This represents a loss of efficiency in the mercaptan production process and an added cost associated with destroying them.

Dialkyl sulfides are occasionally upgraded to obtain the corresponding mercaptans, by means of the following reaction (5) (also known as sulfhydrolysis):

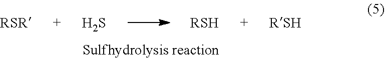

In the case of methyl mercaptan, the sulfhydrolysis reaction is written according to equation (6) below:

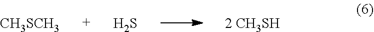

A sulfhydrolysis unit may thus be integrated into a main unit for producing mercaptans obtained from at least one alcohol and H₂S. However, adding a new unit to a pre-existing industrial unit may raise technical integration issues. In particular, the present inventors have observed surprising clogging phenomena in the reactor or downstream of the sulfhydrolysis unit, when the latter is integrated into a main mercaptan production unit.

There is thus a need for a unit for producing mercaptans from alcohol(s) and H₂S incorporating a sulfhydrolysis reaction, which is industrially viable, safe and inexpensive.

One objective of the present invention is to provide an integrated process for preparing mercaptans, notably from at least one alcohol and H₂S, in which the dialkyl sulfide byproducts are treated by sulfhydrolysis, in an industrially viable and safe manner.

Another objective of the present invention is to provide a process for preparing mercaptans, notably from at least one alcohol and H₂S, incorporating a sulfhydrolysis process that is easy to perform, in particular using simple and inexpensive facilities.

Specifically, it has been observed that the preparation of mercaptan(s) from at least one alcohol and H₂S can lead to the formation of dialkyl disulfide impurities (noted DADS and of the type R—S—S—R). Without wishing to be bound by theory, these DADS could be formed according to the following balanced equation (7) (example starting from methanol):

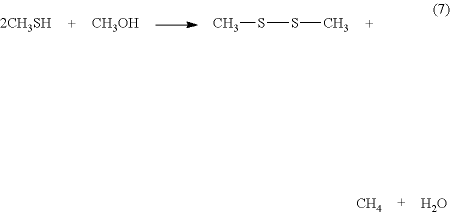

The present inventors have determined that these DADS end up with the dialkyl sulfide(s) and then in the reactor in which the sulfhydrolysis is performed. Over time, they may lead to pressure losses on the catalyst and/or to clogging in this reactor or further downstream in the process. This phenomenon might be explained by coking of the catalyst due to parasitic or secondary reactions of the sulfhydrolysis reaction with the DADS. The sulfur products or impurities formed by such reactions may accumulate and create blockages in industrial facilities, giving rise to obvious safety and production problems. This may be all the more problematic when it comes to recycling the outlet stream from the sulfhydrolysis reactor into the main mercaptan production unit.

In particular, when methyl mercaptan is formed from methanol and H₂S, dimethyl disulfide (DMDS) may be formed secondarily. When this DMDS is present during the sulfhydrolysis reaction, it has been observed that the facilities (in the reactor and downstream of the sulfhydrolysis reactor) are clogged with sulfur impurities.

The present inventors have discovered, surprisingly, that when sulfhydrolysis is performed on a dialkyl sulfide previously separated from the DADS, these phenomena of pressure losses and/or clogging are no longer observed.

Thus, the present invention relates to a process for preparing, preferably continuously, at least one mercaptan, comprising the following steps:
A) H₂S and at least one alcohol are introduced into a first reactor;
B) the H₂S and said at least one alcohol are reacted to obtain an outlet stream comprising at least one mercaptan, at least one dialkyl sulfide and at least one dialkyl disulfide (DADS) and possibly unreacted H₂S;
C) said outlet stream obtained from step B) is separated into:
   a stream F1 comprising the mercaptan(s),
   a stream F2 comprising the dialkyl sulfide(s) and the DADS(s), and
   optionally a stream F3 comprising H₂S;
D) a purification step is performed on stream F2 in order to separate:
   a stream F2′ comprising the dialkyl sulfide(s); and
   the DADS(s);
E) stream F2′ is introduced with H₂S into a second reactor;
F) a sulfhydrolysis reaction of the dialkyl sulfide(s) with H₂S is performed to obtain an outlet stream F4 comprising said mercaptan(s), and possibly unreacted H₂S;
G) optionally, stream F4 obtained from step F) is recycled into step A).

### Sulfides, Disulfides and Mercaptans

The term “sulfide” notably means any organic compound comprising a —C—S—C— function.

The term “disulfide” notably means any organic compound comprising a —C—S—S—C— function.

The term “dialkyl sulfide” notably means a compound of general formula (I) below:

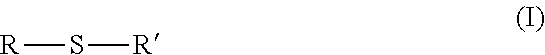

in which R and R′, which may be identical or different, are, independently of each other, a saturated, linear, branched or cyclic, optionally substituted hydrocarbon-based radical.

Preferably, R and R′, which may be identical or different, are, independently of each other, a linear, branched or cyclic alkyl radical containing between 1 and 18 carbon atoms, preferably between 1 and 12 carbon atoms.

R and R′, which may be identical or different, may be chosen, independently of each other, from the group consisting of methyl, ethyl, propyl, butyl, pentyl, hexyl, heptyl, octyl, nonyl, decyl, undecyl and dodecyl (and also positional isomers thereof). Preferably, R and R′, which may be identical or different, may be chosen, independently of each other, from the group consisting of methyl, ethyl, octyl and dodecyl.

Preferably, R and R′ are identical (which corresponds to a symmetrical dialkyl sulfide). Symmetrical dialkyl sulfides in particular have the general formula (II) below:

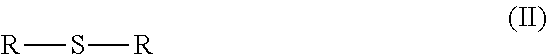

in which R is as defined above.

In particular, the dialkyl sulfides according to the invention are chosen from the group consisting of dimethyl sulfide, diethyl sulfide, dioctyl sulfide, didodecyl sulfide and methyl ethyl sulfide. The dialkyl sulfides according to the invention may be chosen from the group consisting of dimethyl sulfide, di-n-propyl sulfide, di-isopropyl sulfide, di-n-butyl sulfide, di-sec-butyl sulfide and di-isobutyl sulfide. Most particularly preferably, the dialkyl sulfide is dimethyl sulfide (DMS).

The mercaptans according to the invention are notably those corresponding to the sulfhydrolysis of the dialkyl sulfides as defined above. The term “mercaptans” notably means alkyl mercaptans.

In particular, the term “alkyl mercaptan” means a compound of general formula (III) or (IV) below:

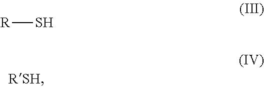

in which R and R′ are as defined for the general formula (I) above.

Particularly preferably, the mercaptan is methyl mercaptan.

In particular, the term “dialkyl disulfide” (also referred to below as DADS) means a compound of the general formula (V) below:

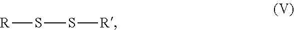

in which R and R′ are as defined for the general formula (I) above.

In particular, the dialkyl disulfides according to the invention are chosen from the group consisting of dimethyl disulfide, diethyl disulfide, dioctyl disulfide, didodecyl disulfide and methyl ethyl disulfide. The dialkyl disulfides according to the invention may be chosen from the group consisting of dimethyl disulfide, diethyl disulfide, dioctyl disulfide and didodecyl disulfide. Most particularly preferably, the dialkyl disulfide is dimethyl disulfide (DMDS).

For steps A) to E), it is understood that when two reagents are introduced into a reactor, they may each be introduced separately into the reactor or may be combined before being introduced into the reactor. The introduction is performed conventionally.

The reactor(s) in which the main reaction and/or the sulfhydrolysis reaction takes place may be fed with fresh H₂S and/or recycled H₂S. The recycled H₂S may be the unreacted H₂S recovered on conclusion of one or more of steps B), C), D) and/or F), preferably on conclusion of steps C) and/or F).

### Step B)—Reaction Between at Least One Alcohol and H₂S to Form a Mercaptan

The reaction between an alcohol and H₂S to form a mercaptan and water is a known reaction, described, for example, in U.S. Pat. Nos. 2,820,062, 7,645,906 B2 and 2,820,831. For example, the reaction may be performed at a temperature of between 200° ° C. and 450° C. and/or at a pressure ranging from a reduced pressure to 100 bar. Generally, a catalyst is present, such as alumina promoted with alkali metals and/or alkaline-earth metals. The H₂S may be present in excess.

Among the reagents, at least one alcohol, preferably one or two alcohols, may be used. Preferentially, only one alcohol is used. The alcohol(s) may notably be chosen from (C₁-C₁₈) or even (C₁-C₁₂) alkane alcohols, and mixtures thereof. In particular, the alcohols may be chosen from the group consisting of methanol, ethanol, octanol, dodecanol and mixtures thereof. Preferably, the alcohol used is methanol.

On conclusion of this step, an outlet stream comprising at least one mercaptan, at least one dialkyl sulfide (as byproduct) and at least one dialkyl disulfide (DADS) (as byproduct or impurity) and possibly H₂S is notably recovered. The outlet stream may also comprise water.

Step C)—Separation of the Mercaptan(s), on the One Hand, from the Dialkyl Sulfide(s) and the DADS, on the Other Hand

Prior to step C), the outlet stream from step B) may undergo one or more purification steps, for example to remove any water and/or H₂S that may be present. This/these purification step(s) may be performed by conventional separation and/or decantation and/or distillation.

In step C), the outlet stream obtained from step B) is separated into:
a stream F1 comprising the mercaptan(s),
a stream F2 comprising the dialkyl sulfide(s) and the DADS(s), and
optionally a stream F3 comprising H₂S.

The separation step C) may be performed via conventional methods, preferably by distillation (notably under reduced pressure). During the distillation, the pressure may be between 1 and 40 bar absolute, and/or the temperature may be between 20° C. and 100° C. at the top of the column, and between 40° C. and 200° C. at the bottom of the column. For example, the distillation may take place at a pressure of between 0.1 bar and 10 bar absolute, notably between 1 and 10 bar absolute.

Preferably, streams F1 and F2 are liquid and/or stream F3 is gaseous. Stream F3 may be totally or partially:
recycled into step A) and/or
recycled into step E) and/or
combined with stream F1 or F2.

### Step D)—Purification of the Dialkyl Sulfides

In step D), a step for purification of stream F2 is notably performed so as to obtain:
a stream F2′ comprising the dialkyl sulfide(s); and
the DADS(s) or a stream F5 comprising the DADS(s).

Step D) is notably a step of separation, on the one hand, of the dialkyl sulfide(s) and, on the other hand, of the DADS(s) and optionally of the heavy impurities present in stream F2. Step D) is more particularly a step of separating dimethyl sulfide from the DMDS present in stream F2.

A stream F2′ enriched in dialkyl sulfide(s) is notably obtained. The DADS or F5 stream may be recovered and optionally purified. The term “stream F2′ enriched in dialkyl sulfide(s)” notably means a stream which comprises a weight percentage of dialkyl sulfide(s) (relative to the total weight of said stream F2′) greater than the weight percentage of dialkyl sulfide(s) relative to the total weight of said stream before said purification step (i.e. of stream F2).

Stream F2 may comprise at least 80%, preferably at least 95%, by weight of dialkyl sulfide(s) relative to the total weight of stream F2. For example, stream F2 comprises between 95% and 99.9% by weight of dialkyl sulfide(s) relative to the total weight of stream F2.

Stream F2 may comprise between 0.1% and 20%, preferably between 0.1% and 5%, by weight of DADS relative to the total weight of stream F2.

Said purification step may correspond to at least one distillation step, or to at least one step of adsorption of the DADS(s) on a porous support (for example on active charcoal), or to at least one step of selective extraction of the DADS(s) using a solvent that is immiscible with said dialkyl sulfide(s) and miscible with said DADS(s), for example water. These various techniques may be combined together.

Entirely preferably, said purification step corresponds to at least one distillation step, preferentially to a single distillation step. According to one embodiment, said purification step consists of a single distillation step.

The pressure during the distillation may be between 0.05 and 75 bar absolute, preferably between 1 and 30 bar absolute, more particularly between 5 and 15 bar absolute, for example approximately 10, 11, 12, 13, 14 or 15 bar absolute.

The distillation temperature may be between 20° C. and 250° C., preferably between 60° C. and 200° C., more preferentially between 100° C. and 180° C.

The column head temperature may be between 20° C. and 250° C., preferably between 60° C. and 200° C., more preferentially between 100° C. and 180° C. In particular, the column head is at a temperature of between 100° C. and 180° C. and at a pressure of between 5 and 15 bar absolute.

The column bottom temperature may be between 50° C. and 300° C., preferably between 100° C. and 250° ° C. In particular, the column bottom temperature is higher than the column head temperature.

Part of stream F2′ may be returned as reflux into the distillation column (stream F6 below). The mass reflux ratio (F6/F2′) in the column may be between 0 and 0.99, preferably between 0 and 0.70.

Preferably, stream F2′ is recovered at the top of the column and the DADS (or stream F5) is recovered at the bottom.

As indicated above, stream F3 may be combined with stream F2, in which case it may undergo the purification step D). In the case of distillation, the H₂S ends up at the top with stream F2′ and may be sent with it to the sulfhydrolysis reactor.

The distillation may be performed in any known type of distillation column. It may be a column with trays (for example trays with caps, trays with valves or perforated trays) or with packing (for example with bulk or structured packing). The distillation may be performed in a tray column, preferably comprising between 5 and 50 trays, more preferentially between 10 and 40 trays, for example between 10 and 30 trays. The distillation may also be performed in a partition column (“DWC” or Divided Wall Column). The partition may be fixed or mobile, for example with structured or bulk packing.

On conclusion of step D), stream F2′ notably comprises less than 1000 ppm (by mass), preferably less than 500 ppm, more preferentially less than 100 ppm or even less than 10 ppm of DADS. In particular, stream F2′ comprises strictly less than 1000 ppm (by mass).

The mercaptan(s) may be recovered from stream F1 and/or stream F4, preferably by recovery from stream F1.

Step F)—Sulfhydrolysis Reaction of Dialkyl Sulfide(s) with H₂S

Stream F2′ is introduced with a stream of H₂S into a second reactor.

The sulfhydrolysis reagents may be in gaseous, liquid or solid form, preferably gaseous or liquid form, under the reaction temperature and pressure conditions.

The sulfhydrolysis reaction temperature may be between 100° C. and 500° C., preferably between 200° C. and 400° ° C., more preferably between 200° C. and 380° ° C. and more preferentially between 250° C. and 380° C.

The sulfhydrolysis reaction may be performed at a pressure of between 50 mbar and 100 bar absolute, preferably between atmospheric pressure (about 1 bar) and 50 bar absolute, and advantageously between 5 and 20 bar absolute.

The H₂S/dialkyl sulfide mole ratio may be between 0.1/1 and 50/1, preferably between 2/1 and 20/1. Preferably, said ratio is between 2/1 and 15/1, more preferably between 2/1 and 8/1, for example between 2/1 and 6/1, such as 4/1.

The flow rate of the dialkyl sulfide into the reactor in which the sulfhydrolysis takes place may be gradual.

The reaction is advantageously performed in the presence of a catalyst. The reagents (dialkyl sulfide(s) and H₂S) may respect a particular contact time with the catalyst. This parameter is expressed with the hourly space velocity equation:

(HSV)=(total CNTP gas flow rate by volume of dialkyl sulfide+H₂S entering)/(volume of catalyst in the reactor).

The HSV may be between 100 and 1200 h⁻¹.

The GHSV (Gas Hourly Space Velocity) may be between 1 and 100 000 h⁻¹, preferably between 100 and 10 000 h 1, more preferentially between 100 and 3000 h⁻¹.

The sulfhydrolysis reaction may take place in any type of reactor, for example fixed-bed tube reactors, multitubular reactors, with microchannels, with a catalytic wall or with a fluidized bed, preferably a fixed-bed tube reactor.

The amount of each reagent supplied to the reactor may vary as a function of the reaction conditions (for example the temperature, the hourly space velocity, etc.) and is determined according to the general knowledge. The hydrogen sulfide may be present in excess.

Any type of catalyst allowing the sulfhydrolysis reaction to be catalyzed may thus be used. Mention may notably be made of catalysts, whether promoted or non-promoted, based on zeolites, alumina (Al₂O₃), silica (SiO₂), titanium dioxide (TiO₂), aluminosilicate, bentonite or zirconia (ZrO₂). These catalysts comprise or may consist of zeolites, alumina (Al₂O₃), silica (SiO₂), titanium dioxide (TiO₂), aluminosilicate, bentonite or zirconia (ZrO₂) and optionally one or more promoters.

The term “promoter” (also known as a “dopant”) notably means a chemical substance or a composition of chemical substances which can modify and notably improve the catalytic activity of a catalyst. For example, the term “promoter” means a chemical substance or a composition of chemical substances for improving the conversion and/or selectivity of the catalyzed reaction relative to the catalyst alone. Such substances are well known, for example alkali metals, nickel (Ni), molybdenum (Mo), cobalt (Co), tungsten (W) or combinations thereof (for example, NiMo and CoMo combinations). It is understood that these promoters may be in their oxide or sulfide form (for example, sodium may be in the oxidized Na₂O form). Preferably, the promoter is chosen from alkali metal oxides, in particular Na₂O.

The term “alkali metal” notably means lithium, sodium, potassium, rubidium and cesium, preferably sodium.

In particular, said catalyst comprises less than 10% by weight of promoter, more preferentially less than 2% by weight of promoter, relative to the total weight of the catalyst. Said catalyst may comprise between 0% and 10% by weight of promoter, preferably between 0% and 2% by weight of promoter, for example between 0.01% and 2% by weight of promoter, relative to the total weight of said catalyst.

The following catalysts may thus be used:
promoted or non-promoted zeolites; preferably zeolites of type X, Y or L, more preferentially zeolites Y;
promoted or non-promoted alumina-based catalysts;
mention may be made in particular of catalysts based on alumina, based on NiMo (nickel/molybdenum) and/or CoMo (cobalt/molybdenum) supported on alumina, based on cadmium sulfide supported on alumina, based on tungsten trisulfide supported on alumina, based on alumina promoted with at least 1% by weight of alkali metal oxide, or else based on non-promoted alumina, for example gamma-alumina (such catalysts are notably described in patent applications WO 2018/035316, WO 2017/210070 and US 2008/0200730);
promoted or non-promoted silica (SiO₂) catalysts;
promoted or non-promoted titanium dioxide (TiO₂) catalysts, (notably as described in patent application FR3101631);
promoted or non-promoted aluminosilicate-based catalysts;
promoted or non-promoted bentonite-based catalysts, for example promoted with at least 1% by weight of an alkali metal oxide, as described in US 2008/0200730; and
promoted or non-promoted zirconia-based catalysts (notably as described in patent application FR 3101631).

Preferably, the catalyst according to the invention is a promoted or non-promoted zeolite of the X, Y or L type, more preferentially of the Y type.

A zeolite is a crystal formed from an aluminosilicate micropore skeleton or support, whose connected void spaces are initially occupied by cations and water molecules.

The zeolites according to the invention in particular have a lattice parameter of between 24.30 and 24.70 Å and/or an Si/Al ratio of between 2.5 and 15. Mention may thus be made of the zeolites sold by the company Axens under the name TCC101®.

In particular, said zeolite comprises less than 10% by weight of alkali metal oxide, more preferentially less than 2% by weight of alkali metal oxide, relative to the total weight of the zeolite. In particular, said alkali metal oxide is sodium oxide (Na₂O).

Most particularly preferably, said catalyst is a Y-type zeolite comprising between 0% and 10%, preferably between 0.01% and 10%, more preferentially between 0.01% and 2% by weight of an alkali metal oxide (preferably Na₂O), relative to the total weight of the zeolite.

The initial zeolite cation, for example sodium, may have been totally or partially replaced with at least one other cation using techniques known for zeolites, for example chosen from the group consisting of (with the exception of the cation to be replaced in the list below): H, Li, Na, K, Mg, Ca, Cs, Ba, La, Zr, Mo, W, Mn, Re, Fe, Ru, Co, Rh, Ni, Pd, Pt, Cu, Zn, Ag, Sn and Ga. These cation exchanges may be performed via conventional techniques.

For example, the zeolites may be treated in ammonium form. This involves calcination in the presence of steam. The zeolite in ammonium form (NH₄⁺) is then converted into protonic form (H⁺) by heating. Steam treatment hydrolyzes the Si—O—Al bonds. Aluminum migrates into the micropore volume in the form of aluminic debris. Simultaneously with the creation of these aluminic or silicoaluminic species, part of the lattice collapses, thus creating a mesoporosity. The silicon in these parts of the lattice is then transported to the vacant sites by the steam. Such techniques are notably explained in the publication by Christine E. A. Kirschhock, Eddy J. P. Feijen, Pierre A. Jacobs, Johan A. Martens; first published: 15 Mar. 2008 https://doi.org/10.1002/9783527610044.hetcat0010 (Part 2. Preparation of Solid Catalysts; 2.3. Bulk Catalysts and Supports; 2.3.5 Hydrothermal Zeolite Synthesis).

The catalysts according to the invention may comprise stabilizers and/or binders. The stabilizers and binders are those conventionally used in the field of catalysts. The catalysts may be pre-activated or pretreated.

### Step G)—Optional Recycling

The outlet stream F4 from the second reactor of step F) may be recycled, partially or entirely, into the first reactor of step A). The H₂S contained in stream F4 may thus be totally or partially recycled. Such recycling notably has the advantage of having only one H₂S inlet for the entire mercaptan production process, for example at the inlet of the sulfhydrolysis reactor.

Thus, the process for the sulfhydrolysis of purified dialkyl sulfides integrated into an industrial facility for the production of mercaptans according to the invention makes it possible to efficiently reprocess the dialkyl sulfide byproducts as products of interest and advantageously to recycle the H₂S and to prevent any clogging and/or pressure losses so as to operate safely and continuously.

The mercaptans produced will be the result of the main reaction and of the sulfhydrolysis reaction, which increases the productivity.

Furthermore, the mercaptans derived from the sulfhydrolysis and the unreacted H₂S can be reintroduced directly (notably without an intermediate purification step) into the main reactor, and without this having any consequence on the reaction between the alcohol(s) and the H₂S. The mercaptans produced by the two reactions (main reaction and sulfhydrolysis reaction) can then be purified and/or recovered in a single place, for example at the outlet of the main reactor. Thus, according to the invention, a simple and efficient process for the upgrading of dialkyl sulfides which is totally integrated into an industrial mercaptan production chain may be obtained. This device is notably easy to implement: it can be readily connected to the main unit and requires only minimal modifications thereof.

The present invention also relates to the use of at least one distillation to purify a stream comprising a dialkyl sulfide of its DADS impurities, prior to performing the sulfhydrolysis of said dialkyl sulfide with H₂S, notably as described above.

The expression “between X and X” includes the limits mentioned, unless mentioned otherwise.

### DESCRIPTION OF THE FIGURES

FIG. 1 schematically shows a methyl mercaptan production unit incorporating a process for the sulfhydrolysis of a purified dimethyl sulfide (DMS).

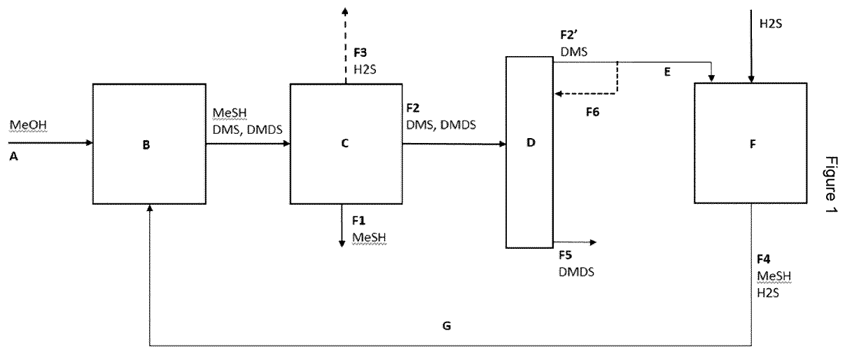

In step A), H₂S and methanol are placed in the reactor in which step B) takes place, to form a stream comprising methyl mercaptan, DMS and DMDS. This stream undergoes separation in step C) to obtain:
a stream F1 comprising methyl mercaptan,
a stream F2 comprising the DMS and the DMDS, and
   an optional stream F3 comprising H₂S;
Stream F2 is distilled to separate the DMS from the DMDS impurity and to obtain a stream F2′ at the top of the column comprising purified DMS. A stream F5 comprising the DMDS is obtained at the bottom of the column. A stream F6, part of F2′, is returned to the distillation column. Stream F2′ is introduced with a stream of H₂S, into a reactor to perform the sulfhydrolysis reaction (step F)). An outlet stream F4 comprising methyl mercaptan and H₂S is obtained. Stream F4 is fully recycled into step A).

The examples below are given for illustrative purposes and do not limit the present invention.

### EXAMPLES

### Example 1: Separation of the DMDS Impurity Prior to the Sulfhydrolysis Reaction

### Test A:

A dimethyl sulfide (DMS) sulfhydrolysis reaction is performed in the presence or absence of DMDS, as follows.

DMS comprising the following (relative to the total weight DMS+DMDS) is introduced into a reactor:
either 0.02% by weight of DMDS;
or 14% by weight of DMDS.

The sulfhydrolysis reaction is performed under the following conditions.

The catalyst used is TCC101® from Axens (catalyst in 1/8 extruded form with an internal radius of 7.7 mm).

It is a Y-type zeolite with a lattice parameter of between 24.30 and 24.70 Å, an Si/Al ratio of between 2.5 and 15 and comprising less than 10% by weight of Na₂O.

The reaction temperature is 340° C. and the pressure is 25 barg.

The H₂S/DMS mole ratio is 30.0.

Result: With DMS comprising 14% by weight of DMDS, clogging was observed in the reactor after a few hours, whereas with DMS comprising 0.02% by weight of DMDS, no clogging was observed after 1000 hours.

This test demonstrates the role of the DMDS impurity in the clogging phenomena.

### Test B:

Before performing the sulfhydrolysis reaction as described in test A, the introduced DMS is first separated or not from the DMDS impurity by distillation.

The distillation conditions are as follows:

A column with a number of trays of between 10 and 20 is used.

The column head pressure is between 5 and 15 barg.

The column head temperature is between 130° ° C. and 140° C.

The column bottom temperature is between 135° ° C. and 150° C.

The reflux flow rate is between 900 kg/h and 1200 kg/h.

### Composition of the Entering Stream:

TABLE 1

| Mass composition of the | Without | With |
|---|---|---|
| stream introduced into | distillation | Distillation |
| the sulfhydrolysis reactor | (F2) | (F2′) |
| % DMS | 98.8% | 99.3% |
| % MeSH | 0.07% | 0.08% |
| % H₂O | 0.60% | 0.60% |
| % DMDS | 0.53% | 0.02% |

Without prior distillation, clogging occurs after 100 h of running the sulfhydrolysis. With prior distillation, no clogging is observed after 1000 h.

1. A process for preparing at least one mercaptan, comprising the following steps:
A) H₂S and at least one alcohol are introduced into a first reactor;
B) the H₂S and said at least one alcohol are reacted to obtain an outlet stream comprising at least one mercaptan, at least one dialkyl sulfide and at least one dialkyl disulfide (DADS) and possibly unreacted H₂S;
C) said outlet stream obtained from step B) is separated into:
a stream F1 comprising the mercaptan(s),
a stream F2 comprising the dialkyl sulfide(s) and the DADS(s), and
optionally a stream F3 comprising H₂S;
D) a purification step is performed on stream F2 in order to separate:
a stream F2′ comprising the dialkyl sulfide(s); and
the DADS(s);
E) stream F2′ is introduced with H₂S into a second reactor;
F) a sulfhydrolysis reaction of the dialkyl sulfide(s) with H₂S is performed to obtain an outlet stream F4 comprising said mercaptan(s), and possibly unreacted H₂S;
G) optionally, stream F4 obtained from step F) is recycled into step A).

2. The preparation process according to claim 1, in which said step D) corresponds to at least one distillation step, or to at least one step of adsorption of the DADS(s) on a porous support, or to at least one step of selective extraction of the DADS(s) using a solvent that is immiscible with said dialkyl sulfide(s) and miscible with said DADS(s).

3. The preparation process according to claim 1, in which said step D) corresponds to at least one distillation step, preferentially to a single distillation step.

4. The preparation process according to claim 3, in which, in the distillation step D), the pressure is between 0.05 and 75 bar absolute, preferably between 1 and 30 bar absolute, more particularly between 5 and 15 bar absolute.

5. The preparation process according to claim 3, in which, in the distillation step D), part of stream F2′ may be returned to the distillation column as reflux.

6. The preparation process according to claim 3, in which, in the distillation step D):
the column head temperature is between 20° C. and 250° C., preferably between 60° C. and 200° C., more preferentially between 100° ° C. and 180° C.; and
the column bottom temperature is between 50° C. and 300° C., preferably between 100° C. and 250° ° C.

7. The preparation process according to claim 1, in which step F) is performed in the presence of a catalyst chosen from promoted or non-promoted catalysts based on zeolites, alumina (Al₂O₃), silica (SiO₂), titanium dioxide (TiO₂), aluminosilicate, bentonite or zirconia (ZrO₂); preferably, the catalyst is a zeolite.

8. The preparation process according to claim 1, in which, in step F), the H₂S/dialkyl sulfide mole ratio is between 0.1/1 and 50/1, preferably between 2/1 and 20/1, more preferentially between 2/1 and 8/1.

9. The preparation process according to claim 1, in which, in step G), all of the stream F4 is recycled into step A).

10. The preparation process according to claim 1, in which the alcohol of step A) is methanol.
